(19) United States
(12) Patent Application Publication
(10) Pub. No.: US 2014/0207247 A1
(43) Pub. Date: Jul. 24, 2014

(54) Implant and implant apparatus

(71) Applicant: TERUMO KABUSHIKI KAISHA, Tokyo (JP)

(72) Inventors: Atsuhiko NOGAWA, Kanagawa (JP); Shigeki ARIURA, Kanagawa (JP); Masakatsu KAWAURA, Kanagawa (JP)

(73) Assignee: TERUMO KABUSHIKI KAISHA, Tokyo (JP)

(21) Appl. No.: 14/223,288

(22) Filed: Mar. 24, 2014

Related U.S. Application Data
(63) Continuation of application No. PCT/JP2012/070552, filed on Aug. 10, 2012.

(30) Foreign Application Priority Data
Sep. 28, 2011 (JP) 2011-211976

(51) Int. Cl. A61F 2/04

(52) U.S. Cl. CPC A61F 2/042
USPC 623/23.66

(57) ABSTRACT

An implant is an implantable device for the treatment of urinary incontinence in women, that is, a device that supports the urethra in a state where the urethra is pulled in a direction such that the urethra is separated from the vaginal wall. The implant includes a pad which can be recovered from a constrained state by a self-recovery force and which has a plate-shaped appearance in the recovered state; and at least one string that passes through the pad to fix the pad in a state where the pad supports the urethra. The pad has a mesh-like shape with a plurality of openings. When the pad is fixed to the living body so as to support the urethra, the string passes through mesh opening of the pad and a fixing portion that is fixed to the living body is provided in one end portion of the string.

### CROSS REFERENCES TO RELATED APPLICATIONS

This application claims priority as a continuation application under 35 U.S.C. 120 to International Application No. PCT/JP2012/070552 filed on Aug. 10, 2012, designating the U.S., and which claims priority to Japanese Application No. 2011-211976 filed on Sep. 28, 2011, the entire content of both of which is incorporated herein by reference.

### TECHNICAL FIELD

The disclosure herein relates to an implant and an implant apparatus.

### BACKGROUND DISCUSSION

A patient suffering from urinary incontinence, and in particular, suffering from stress urinary incontinence, experiences urine leakage caused by intra-abdominal pressure occurring from normal exercise, laughing, coughing, sneezing or the like.

The fact that the pelvic floor muscles which support the urethra become weak after childbirth is cited as one of the causes.

There is a surgical treatment that is effective in treating urinary incontinence. During such surgical treatment, a tape-shaped implant referred to as a “sling” is indwelled inside the body to support the urethra (for example, as disclosed in JP-A-2010-099499).

In order to indwell the sling inside the body, an operator incises the vagina, dissects tissue in the area between the urethra and the vagina, and communicates the dissected region with the outside using a puncture needle or the like through an obturator foramen.

In such a state, the sling is indwelled inside the body.

However, there is a problem in that the procedure is very invasion and the patient bears a great burden if the sling is indwelled using the methods known in the related art.

In addition, there is a concern that the urethra or the like may be damaged in the course of the surgical procedure.

### SUMMARY

The disclosure here provides an implantable implant and an implant apparatus by which a patient suffering from urinary incontinence receives treatment with minimal burden on the patient and high patient safety.

An exemplary embodiment of the disclosure is directed to an implantable implant for the treatment of urinary incontinence in women, wherein the implant includes a pad, the shape of which can be recovered from a constrained state and which has a plate-shaped appearance in the recovered state; and a fixing member that passes through the pad to fix the pad to a living body in a state where the pad supports the urethra.

In the implant according to an exemplary embodiment of the disclosure, the pad preferably recovers from a folded state, a rolled-up state or a compressed state by a self-recovery force.

In the implant according to an exemplary embodiment of the disclosure, it is preferable that the pad have a mesh-like shape and the fixing member pass through meshes of the pad when the fixing member fixes the pad.

Further, according to an exemplary embodiment of the disclosure, the fixing member preferably is a long object that is flexible. A fixing portion that is fixed to the living body is preferably provided in at least one end portion of the long object. The fixing portion is a portion that punctures the living body and the fixing portion of the long object preferably has a greater resistance at the time the long object is pulled out from the living body compared to other portions of the long object.

In the implant according to an exemplary embodiment of the disclosure, the pad is preferably configured to be pulled up and fixed to a side of the urethra from a side of the vaginal wall by the tension from the long object.

In addition, the disclosure here is directed to an implant apparatus which includes the implant according to the aforesaid exemplary embodiment of the disclosure; and a tubular body that is used to move the pad to a target portion in a state where the shape of the pad is constrained.

In the implant apparatus according to an exemplary embodiment of the disclosure, the tubular body is preferably used to move the fixing member to a target portion.

It is preferable that the implant apparatus according to an exemplary embodiment of the disclosure further includes a positioning member that positions a distal portion of the tubular body with respect to the pad. It is also preferable that the positioning member be a string-like body of which one end side is fixed to the pad and that the positioning member be inserted into the tubular body for use.

Still further, in an exemplary embodiment of the disclosure, it is preferable that one end side of the string-like body is fixed to a center portion of the pad, and that the string-like body is configured to position the distal portion of the tubular body with respect to the center portion of the pad.

According to an exemplary embodiment of the disclosure, it is also preferable that the positioning member be a string-like body that has a foldback portion which is folded back, retains the pad in the foldback portion, and is inserted into the tubular body for use.

Further, it is preferable that the foldback portion of the string-like body retain the center portion of the pad, and that the string-like body be configured to position the distal portion of the tubular body with respect to the center portion of the pad.

### BRIEF DESCRIPTION OF DRAWINGS

These and other features and advantages of the disclosure will become more readily apparent to those skilled in the art upon reading the following detailed description, in conjunction with the appended drawings in which:

FIG. 1 illustrates a guide apparatus of an implant apparatus according to a first exemplary embodiment of the disclosure herein.

FIG. 2 describes the sequence of an operation of the implant apparatus according to the first exemplary embodiment of the disclosure herein.

FIG. 3 describes the sequence of the operation of the implant apparatus according to the first exemplary embodiment of the disclosure herein.

FIG. 4 describes the sequence of the operation of the implant apparatus according to the first exemplary embodiment of the disclosure herein.

FIG. 5 describes the sequence of the operation of the implant apparatus according to the first exemplary embodiment of the disclosure herein.

FIG. 6 describes the sequence of the operation of the implant apparatus according to the first exemplary embodiment of the disclosure herein.

FIG. 7 describes the sequence of the operation of the implant apparatus according to the first exemplary embodiment of the disclosure herein.

FIG. 8 describes the sequence of the operation of the implant apparatus according to the first exemplary embodiment of the disclosure herein.

FIG. 9 describes the sequence of the operation of the implant apparatus according to the first exemplary embodiment of the disclosure herein.

FIG. 10 describes the sequence of the operation of the implant apparatus according to the first exemplary embodiment of the disclosure herein.

FIG. 11 describes the sequence of the operation of the implant apparatus according to the first exemplary embodiment of the disclosure herein.

FIG. 12 describes the sequence of the operation of the implant apparatus according to the first exemplary embodiment of the disclosure herein.

FIG. 13 describes the sequence of the operation of the implant apparatus according to the first exemplary embodiment of the disclosure herein.

FIG. 14 is a cross-sectional view illustrating an inner tube in an implant apparatus according to a second exemplary embodiment of the disclosure herein.

FIG. 15 is a cross-sectional view illustrating the inner tube and a needle in the implant apparatus according to the second exemplary embodiment of the disclosure herein.

FIG. 16 is a cross-sectional view illustrating an example of another configuration of the inner tube in the implant apparatus according to the second exemplary embodiment of the disclosure herein.

FIG. 17 illustrates a pad and a string in an implant apparatus according to a third exemplary embodiment of the disclosure herein.

### DETAILED DESCRIPTION

Preferred embodiments of an implant and an implant apparatus according to the disclosure will be described in detail with reference to the accompanying drawings.

### First Embodiment

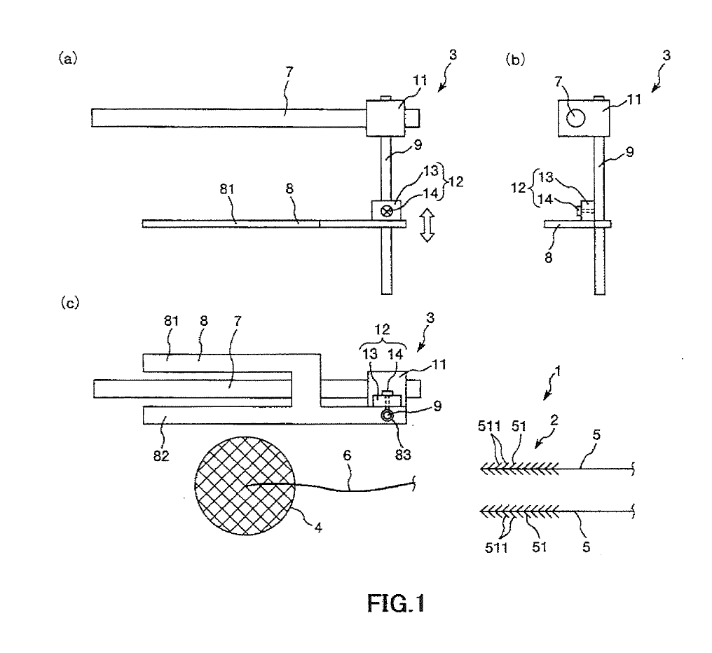

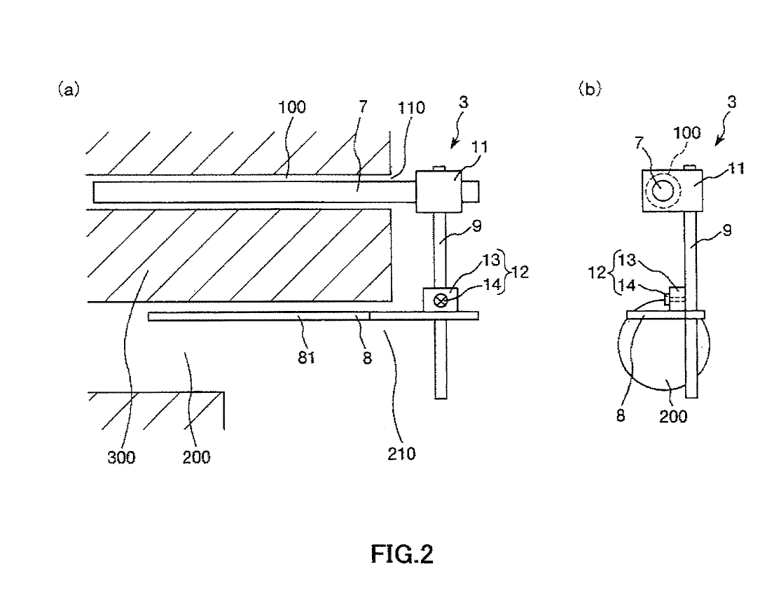

FIG. 1 illustrates a guide apparatus of the implant apparatus according to a first exemplary embodiment of the disclosure, with FIG. 1(a) being a side view, FIG. 1(b) being a rear view, and FIG. 1(c) being a bottom view of the guide apparatus, with the implant and strings further illustrated therein. FIGS. 2 to 13 describe a sequence of operation of the implant apparatus according to the first exemplary embodiment of the disclosure.

Hereinafter, description will be given on the premise that in FIGS. 1(a) to 13(a), the left side indicates a “distal end”, the right side indicates a “proximal end”, the upper side indicates a “top”, and the lower side indicates a “bottom”.

An implant apparatus 1 as illustrated in the drawings is an apparatus that is used for the treatment of urinary incontinence in women.

As shown by way of illustration in the drawings, the implant apparatus 1 includes an implant 2 (a device indwelled inside a living body) and a guide apparatus 3 used to indwell the implant 2 inside the body.

The implant 2 is an implantable device for the treatment of urinary incontinence in women, that is, a device that supports the urethra in a state where the urethra is pulled in a direction such that the urethra is separated from the vaginal wall.

As illustrated in FIG. 1, the implant 2 includes a pad 4, the shape of which can be recovered from a constrained state to an unconstrained state and which has a generally flat, plate-shaped appearance in the recovered state; and two strings (string-like bodies) 5 as fixing members that pass through the pad 4 to fix the pad 4 in a state whereby the pad 4 supports the urethra.

The pad 4 is elastically deformable, and the shape of the pad 4 returns to an original shape from a constrained state by a self-recovery force, that is, by an elastic force.

For example, a folded state, rolled-up state, compressed state or the like of the pad 4 is cited as a state where the shape of the pad 4 is constrained.

The pad 4 has a mesh-like shape in the exemplary embodiment. That is, the pad 4 has a plurality of openings. The percentage of the openings in the pad 4 is not particularly limited in a free state where an external force is not applied to the pad 4, but the percentage is preferably in a range of approximately 30% to 95% and is more preferably in a range of approximately 50% to 90%.

Accordingly, the shape of the pad 4 can be reliably recovered from a constrained state by a self-recovery force.

By way of example, the pad 4 can be braided into a mesh-like shape (lattice shape including open interstices) through the intersection of line-shaped bodies, such that the pad 4 can have the configuration of a braided body.

In such a configuration, examples of the line-shaped bodies may include a body with a circular cross-sectional shape, a body with a flat cross-sectional shape, that is, a belt-shaped (ribbon-shaped) body, or the like.

When the line-shaped body has a circular cross-sectional shape, the diameter thereof is preferably in a range of approximately 0.3 mm to 3 mm and, more preferably, in a range of approximately 0.5 mm to 2 mm.

In addition, when the line-shaped body has a belt-shape, it is preferable that the width thereof be in a range of approximately 2 mm to 10 mm and the thickness thereof be in a range of approximately 0.3 mm to 1.5 mm.

In addition, the shape profile of the pad 4 is a circular shape in the exemplary embodiment as illustrated in FIG. 1(c).

The outer diameter of the pad 4 is not particularly limited, but the outer diameter thereof is preferably in a range of approximately 3 mm to 30 mm and, more preferably, in a range of approximately 5 mm to 20 mm.

In addition, the material of the pad is not particularly limited, and, for example, various types of resin material with biocompatibility or the like can be used for fabrication of the pad.

The mesh-like pad 4 is not limited to a braided body of course, and, for example, the pad 4 can be configured to have a plurality of openings in a plate-shaped member. In addition, the pad is not limited to a mesh-like body and may, alternatively, be formed without mesh openings.

In addition, the shape profile of the pad 4 is not limited to a circular shape, and, for example, a rectangular shape, a polygonal shape such as a pentagonal shape, an elliptical shape or the like are all possible shape profiles for the pad 4.

The strings 5 will be described below, noting that since all of the strings 5 have the same configuration, only one of the strings 5 will be representatively described hereinafter.

When the pad 4 is fixed to the living body, the string 5 passes through meshes of the pad 4, and a fixing portion 51 that is fixed to the living body is provided in one end portion of the string 5.

The fixing portion 51 is a portion that punctures the living body. The fixing portion 51 is configured to have resistance greater than portions other than the string 5 of the fixing portion 51 when pulled out from the living body.

In the exemplary embodiment illustrated herein, the fixing portion 51 is configured to have a plurality of V-shaped unit fixing portions 511. Each of the unit fixing portions 511 is arranged side by side at preferably equal intervals along a longitudinal direction of the string 5.

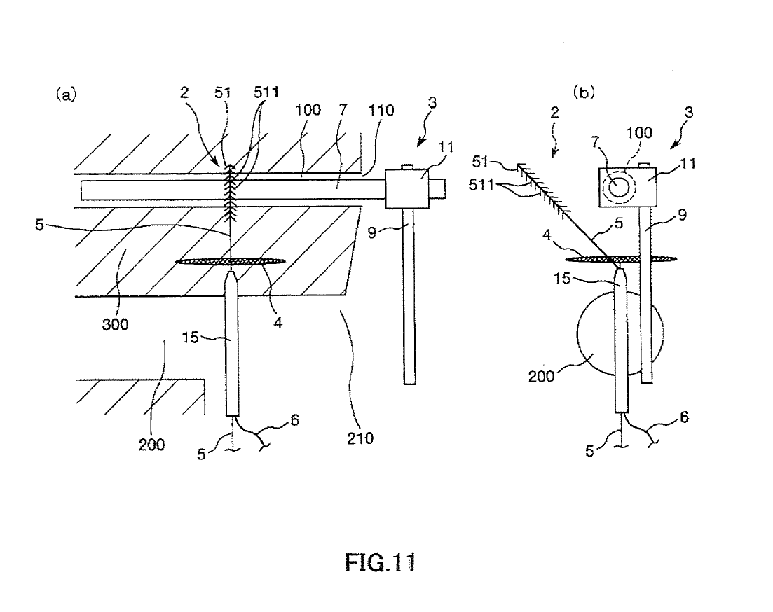

Note that a diverged side of the “V” shape in each of the unit fixing portions 511 is set to face a direction opposite a direction in which the string 5 moves forward when the string 5 punctures the living body (refer to FIGS. 11 to 13).

Each of the strings 5 pulls up and fixes the pad 4 to a side of the urethra from a side of the vaginal wall by the tension from the string 5 in a state where the pad 4 is attached to biological tissues in the periphery of the urethra.

Accordingly, the urethra is pulled in a direction such that the urethra is separated from the vaginal wall and is supported by the pad 4.

In addition, material for each of the strings 5 or a string 6 which will be described later is not particularly limited, and, for example, various types of resin material, fibers with biocompatibility or the like can be used as the material from which the strings are formed.

In the embodiment disclosed here, the implant 2 has two of the strings 5 but the number of the strings 5 is not limited to two and, for example, the implant 2 may have only one string, or more than two strings. In the case of only one string, fixing portions that are fixed to the living body are provided in both end portions of the single string.

Further, the fixing member is not necessarily limited to the string 5. For example, other long objects that are flexible such as a cord or a belt can be used as the fixing member.

The guide apparatus 3 will now be described. As illustrated in each drawing, the guide apparatus 3 has a urethral-insertion portion 7 that is inserted into the urethra; a vaginal insertion portion 8 that is inserted into the vagina; an interlock portion 9 that interlocks the urethral-insertion portion 7 with the vaginal insertion portion 8; a sheath 15 that is a tubular body used to move the pad 4 to a target portion in a state where the shape of the pad 4 is constrained; a string (string-like body) 6 that is a positioning member which positions a distal portion of the sheath (tubular body) 15 with respect to the pad 4; a needle 16 that moves the strings 5 to target portions, respectively; and a needle 17 that guides the sheath 15 when the sheath 15 moves to a target portion.

Each of the urethral-insertion portion 7 and the interlock portion 9 preferably has a bar shape, and a supporting portion 11 is fixed to the right end portion of the urethral-insertion portion 7 as shown in FIG. 1(a).

The supporting portion 11 is fixed to the upper end portion of the interlock portion 9 in such a manner that the interlock portion 9 is orthogonal to the urethral-insertion portion 7.

The interlock portion 9 is arranged below the urethral-insertion portion 7 as illustrated in FIG. 1(c).

The vaginal insertion portion 8 has a plate shape, and the left side thereof, relative to FIG. 1(a), is diverged into two stages to form two protrusion pieces 81 and 82 that protrude in a direction of the distal end.

In the configuration which is illustrated in FIG. (c), the protrusion piece 81 forms an L shape, and the protrusion piece 82 forms a rectangular shape.

A space between the protrusion pieces 81 and 82 is a space for the raised vaginal wall. That is, when the vaginal insertion portion 8 is pushed against the vaginal wall, the vaginal wall is raised to protrude from the space between the protrusion pieces 81 and 82.

The vaginal insertion portion 8, that is, the protrusion pieces 81 and 82 are arranged in parallel with the urethral-insertion portion 7.

In addition, an opening 83 is formed in the right end portion of the vaginal insertion portion 8 relative to FIG. 1(a), and the interlock portion 9 is inserted through the opening 83.

Accordingly, the vaginal insertion portion 8 can move along the interlock portion 9 in a direction orthogonal to the interlock portion 9, and thus a gap between the vaginal insertion portion 8 and the urethral-insertion portion 7 can be adjusted.

A lock portion 12 is provided in the vaginal insertion portion 8 to switch between a state where the vaginal insertion portion 8 can move with respect to the interlock portion 9 and a state where movement of the vaginal insertion portion 8 is hindered.

The lock portion 12 has a female screw portion 13 configured such that a male screw 14 can be screwed into the female screw portion 13. The female screw portion 13 is fixed in the vicinity of the opening 83 of the vaginal insertion portion 8.

When the male screw 14 is rotated in a predetermined direction, the distal end of the male screw 14 comes into press contact with the interlock portion 9 to hinder the vaginal insertion portion 8 from moving.

In addition, when the male screw 14 is rotated in a direction opposite to the predetermined direction, the distal end of the male screw 14 is separated from the interlock portion 9 to thus allow the vaginal insertion portion 8 to move.

As best shown in FIG. 1(c), one end portion of the string 6 is fixed to a center portion of the pad 4 of the implant 2, and the string 6 is inserted into the sheath 15 for use.

As described later, the string 6 positions the distal portion of the sheath 15 with respect to the center portion of the pad 4.

The outer diameter of the distal portion of the sheath 15 gradually reduces in a direction of the distal end. Accordingly, the sheath 15 can easily and smoothly puncture the vaginal wall.

The sheath 15 is used to move not only the pad 4 but also two of the strings 5 to the target portions, respectively, as described later.

The sheath 15 may also be formed to accommodate the pad 4 in the distal portion thereof in a state where the shape of the pad 4 is constrained.

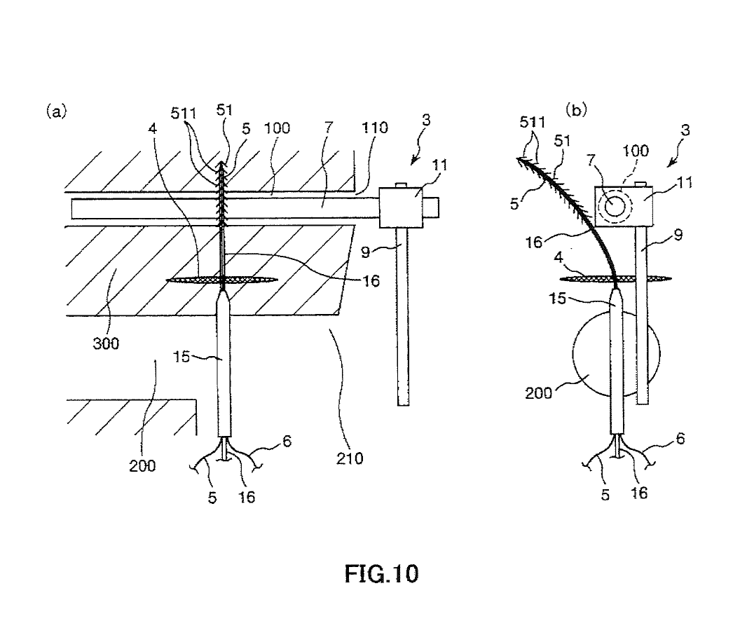

In the exemplary embodiment, the needle 16 is formed into a shape in which a distal portion thereof is curved relative to the middle thereof (refer to FIG. 10). That is, the distal portion of the needle 16 is curved relative to the middle thereof in a free state where an external force is not applied thereto.

A retention portion (not shown) is formed in the distal portion of the needle 16 to separably retain the strings 5.

The needle 16 is inserted into the sheath 15 to move two of the strings 5 to the target portions, respectively.

Note that each of the needles 16 and 17 may be a solid body, or may have a tubular shape.

An operation sequence of the implant apparatus 1 will be described below.

First, as illustrated in FIG. 2, the gap between the urethral-insertion portion 7 and the vaginal insertion portion 8 is adjusted to fit in with a gap between a urethral orifice 110 and a vaginal opening 210 of a patient.

The urethral-insertion portion 7 of the guide apparatus 3 of the implant apparatus 1 is inserted into the urethra 100 through the urethral orifice 110, and the vaginal insertion portion 8 is inserted into a vagina 200 through the vaginal opening 210.

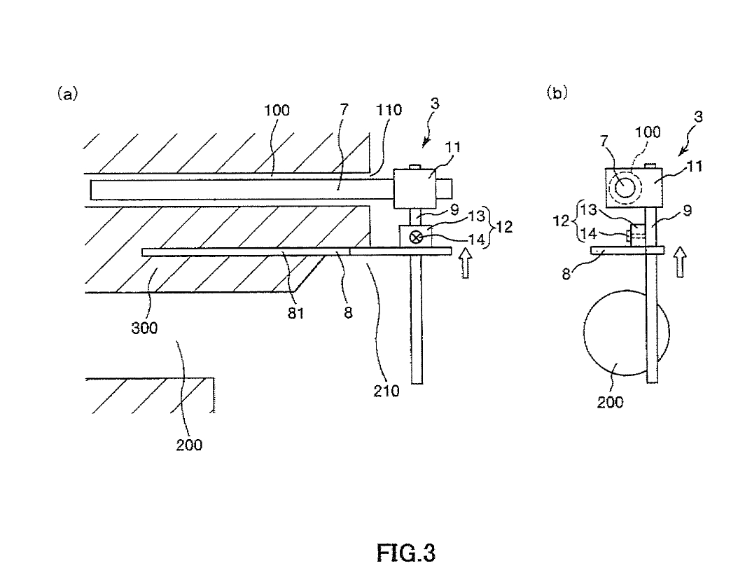

Subsequently, as illustrated in FIG. 3, the male screw 14 of the lock portion 12 is rotated in a loosening direction to allow the vaginal insertion portion 8 to move with respect to the interlock portion 9, and the vaginal insertion portion 8 is moved upward with respect to the interlock portion 9 as shown in FIG. 3(a).

Accordingly, a portion of the vaginal wall 300 is raised from the space between the protrusion pieces 81 and 82 of the vaginal insertion portion 8 to protrude downward as illustrated in FIG. 3(a).

The male screw 14 of the lock portion 12 is rotated in a tightening direction, and the vaginal insertion portion 8 is hindered from moving with respect to the interlock portion 9.

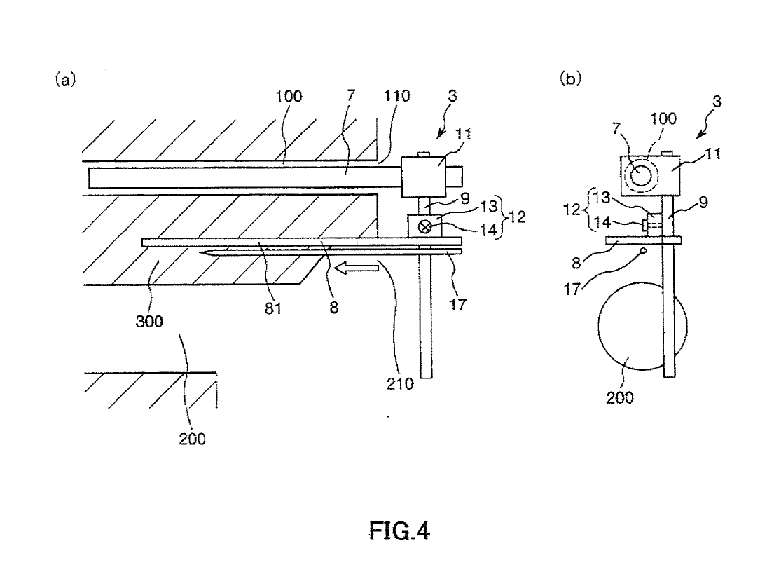

Subsequently, as illustrated in FIG. 4, the needle 17 punctures the raised vaginal wall 300 to move to a target portion in a direction of the distal end, that is, until a distal portion of the needle 17 is positioned to insert the pad 4 of the implant 2.

At this time, the vaginal insertion portion 8 is used to guide the needle 17, and the needle 17 is moved along the vaginal insertion portion 8.

Accordingly, since the needle 17 moves in the direction of the distal end while being in parallel to the urethral-insertion portion 7 that is arranged in parallel to the vaginal insertion portion 8, the needle 17 can be prevented from puncturing the urethra 100.

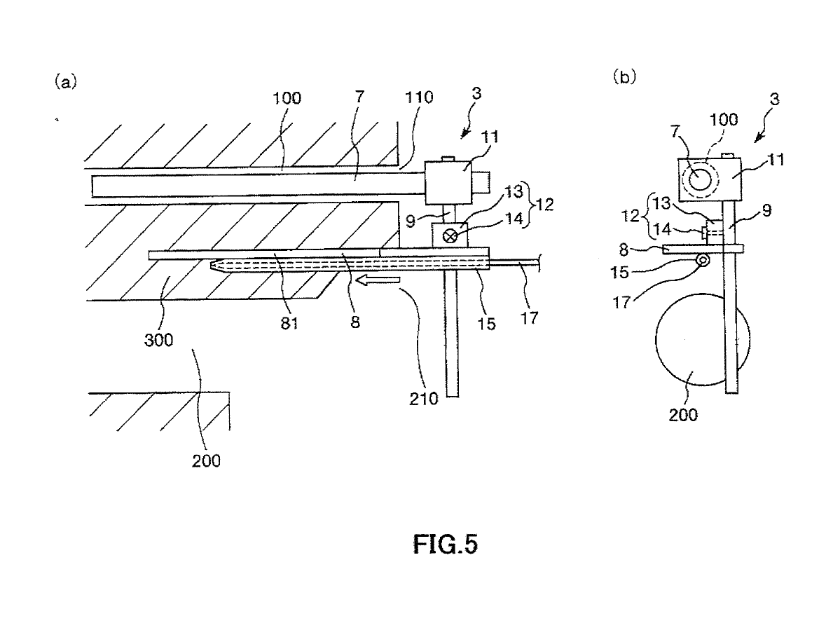

Thereafter, as illustrated in FIG. 5, the sheath 15 covers the needle 17 from a proximal portion of the needle 17 and is moved along the needle 17 in the direction of the distal end.

At this time, the sheath 15 is moved until the distal portion of the sheath 15 coincides with the distal portion of the needle 17, that is, until the distal portion of the sheath 15 is positioned to insert the pad 4.

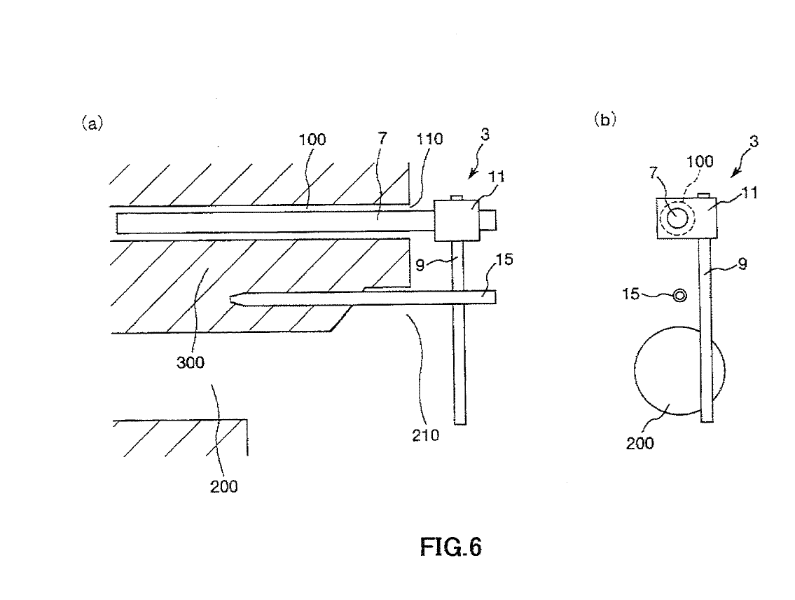

As illustrated in FIG. 6, the needle 17 is then removed.

In addition, the male screw 14 of the lock portion 12 is rotated in the loosening direction to allow the vaginal insertion portion 8 to move with respect to the interlock portion 9, and the vaginal insertion portion 8 is moved downward with respect to the interlock portion 9 so as to detach from the interlock portion 9 as shown in FIG. 6(a).

The pad 4 in a rolled-up state is then inserted into the sheath 15 using a fixture (not shown) until the pad 4 reaches the distal portion of the sheath 15. Note that portions of the string 6, which are opposite the pad 4, protrude outside from the proximal end of the sheath 15.

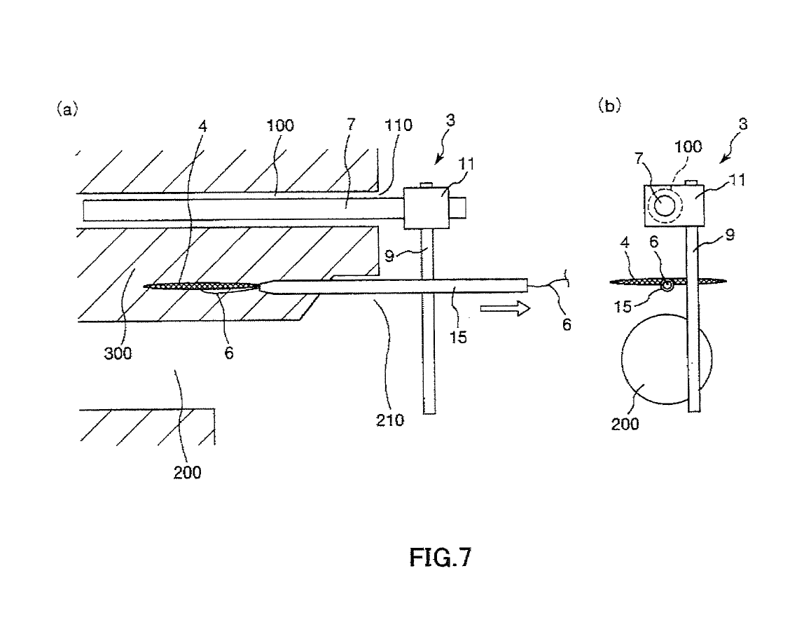

Subsequently, as illustrated in FIG. 7, while the pad 4 is pushed using the fixture, the sheath 15 is moved in a direction of the proximal end.

Accordingly, the pad 4 is released into the vaginal wall 300 from the distal end of the sheath 15.

Since it is relatively easy to dissect the area between the urethra 100 and the vagina 200, when the pad 4 is released into the vaginal wall 300, the shape of the pad 4 returns to an original shape from the rolled-up shape by a self-recovery force, and at this time, the pad 4 dissects the area between the urethra 100 and the vagina 200 and is arranged between the urethra 100 and the vagina 200.

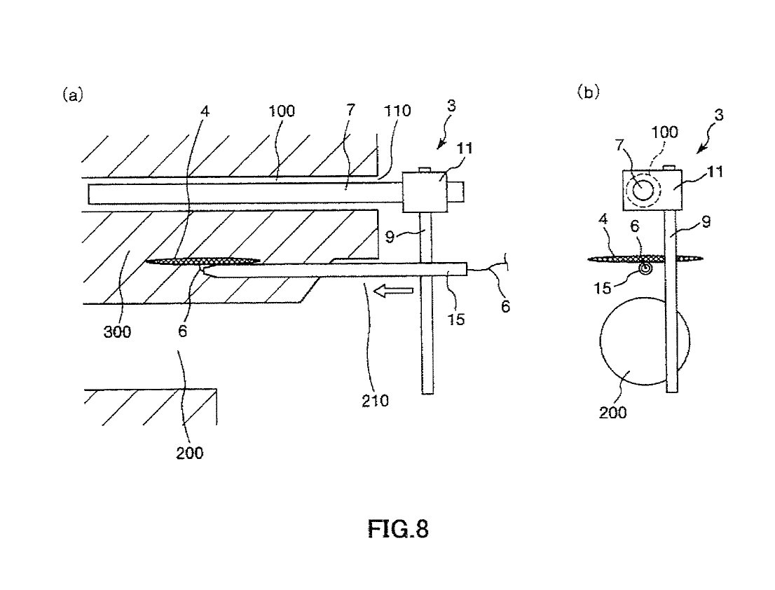

As illustrated in FIG. 8, while the string 6 is pulled in the direction of the proximal end, the sheath 15 is moved in the direction of the distal end.

Thus, the sheath 15 is moved until the distal end of the sheath 15 is positioned at an end portion of the string 6 that is fixed to the center portion of the pad 4, that is, until the distal end of the sheath 15 is disposed at, i.e., adjacent, the center portion of the pad 4.

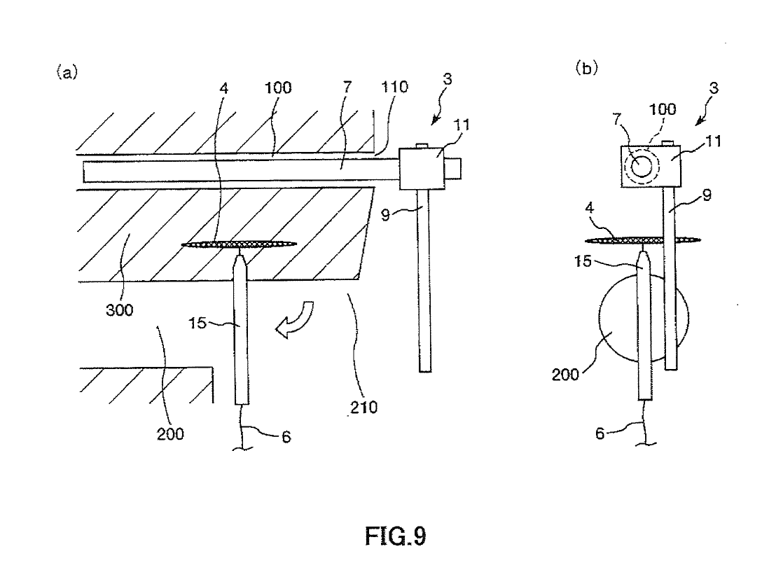

Subsequently, as illustrated in FIG. 9, while the string 6 is pulled in the direction of the proximal end, the sheath 15 and the string 6 are rotated 90° clockwise around the distal end of the sheath 15 as shown in FIG. 9(a).

Accordingly, the sheath 15 is orthogonal to the urethra 100.

Next, as illustrated in FIG. 10, the retention portion (not shown) of the distal portion of the needle 16 retains the fixing portion 51 of one of the pair of strings 5. A needle-axis rotational position of the needle 16 is set in such a manner that the distal portion of the needle 16 faces the upper left direction as shown in FIG. 10(b), and the needle 16 is inserted into the sheath 15 to be moved in the direction of the distal end.

Since the needle 16 is elastic, the distal portion thereof becomes a linear shape from a curved shape to be inserted into the sheath 15.

When the needle 16 is moved in the direction of the distal end, the distal portion of the needle 16 and the fixing portion 51 of the string 5 protrude from the distal end of the sheath 15 to pass through the meshes of the pad 4.

In addition, when the needle 16 protrudes from the distal end of the sheath 15, the shape of the distal portion thereof returns to an original shape by a self-recovery force.

Accordingly, as illustrated in FIG. 10(b), the distal portion of the needle 16 avoids the urethra 100 and advances in the upper left direction in a curved form. Thus, the needle 16 can be prevented from puncturing the urethra 100.

The needle 16 is moved until the fixing portion 51 of the string 5 reaches a target portion. In this way, the fixing portion 51 of the string 5 punctures the living body.

Thereafter, as illustrated in FIG. 11, the needle 16 is removed.

At this time, each of the unit fixing portions 511 of the fixing portion 51 of the string 5 punctures the living body, the fixing portion 51 thereof is fixed to the living body, and thus the fixing portion 51 can be prevented from falling off the living body. Hence, the fixing portion 51 of one string 5 is fixed to the living body.

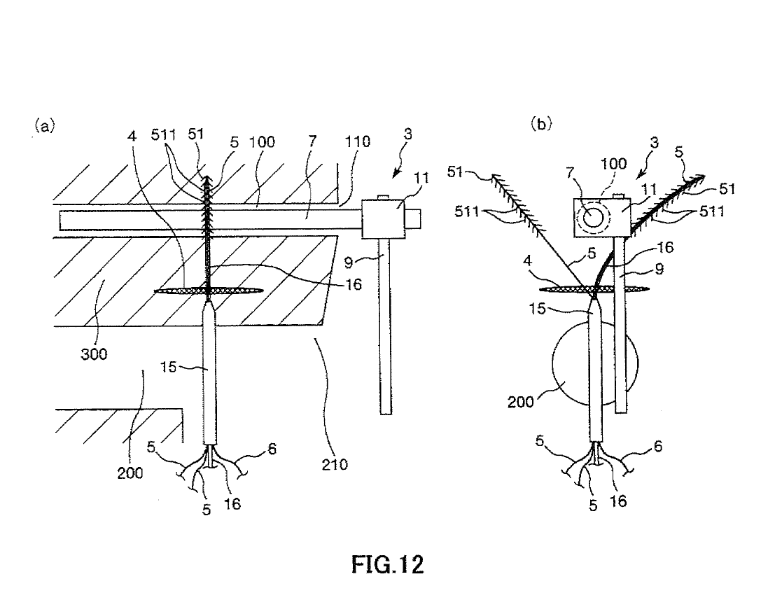

Subsequently, as illustrated in FIG. 12, the retention portion (not shown) of the distal portion of the needle 16 retains the fixing portion 51 of the other string 5. A needle-axis rotational position of the needle 16 is set in such a manner that the distal portion of the needle 16 faces the upper right direction relative to FIG. 12(b), and the needle 16 is inserted into the sheath 15 to be moved in the direction of the distal end.

When the needle 16 is moved in the direction of the distal end, the distal portion of the needle 16 and the fixing portion 51 of the string 5 protrude from the distal end of the sheath 15 to pass through the meshes of the pad 4.

The shape of the distal portion thereof returns to an original shape by a self-recovery force.

Accordingly, as illustrated in FIG. 12(b), the distal portion of the needle 16 avoids the urethra 100 and advances in the upper right direction in FIG. 12(b) in a curved form. Hence, the needle 16 can be prevented from puncturing the urethra 100.

The needle 16 is moved until the fixing portion 51 of the string 5 reaches a target portion. In this way, the fixing portion 51 of the string 5 punctures the living body.

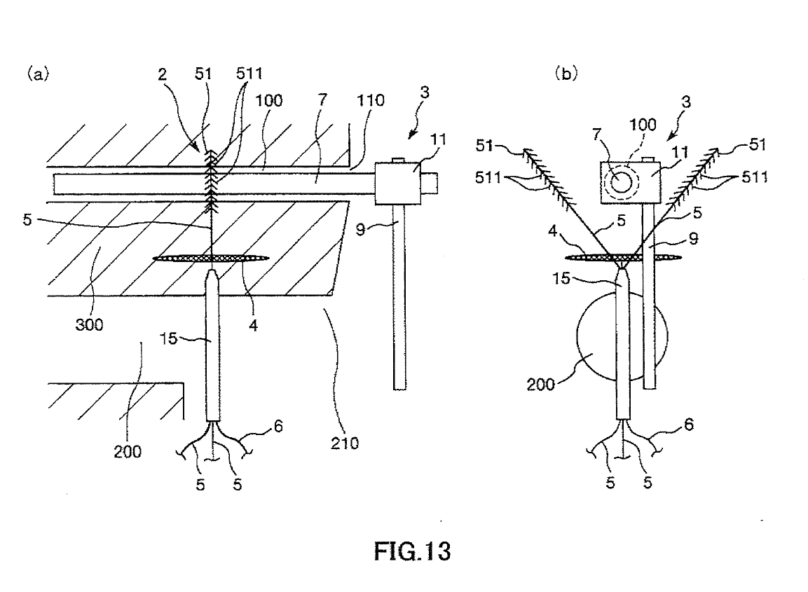

Subsequently, as illustrated in FIG. 13, the needle 16 is removed.

At this time, each of the unit fixing portions 511 of the fixing portion 51 of the string 5 punctures the living body, the fixing portion 51 thereof is fixed to the living body, and thus the fixing portion 51 can be prevented from falling off the living body. In this way, the fixing portion 51 of the other string 5 is thus fixed to the living body.

Subsequently, an apparatus (not shown) combines each one of the strings 5 and the string 6 to form a knot (not illustrated). For example, a clinch knot or the like in which a knot is unidirectionally movable is used as a method of tying a knot. The knot is moved upward in FIG. 13.

Thus, the pad 4 and the knot are moved upward in FIG. 13, and the pad 4 is attached to the biological tissues in the periphery of the urethra.

At this time, an operator adjusts a tightening state of each of the strings 5 by moving the knot. In this way, the pad 4 is pulled up and fixed to a side of the urethra 100 from a side of the vaginal wall 300 by the tension from each of the strings 5 in a state where the pad 4 is attached to the biological tissues in the periphery of the urethra 100.

Accordingly, the urethra 100 is pulled in a direction such that the urethra 100 is separated from the vaginal wall 300 and is supported by the pad 4.

Subsequently, the sheath 15 is removed, unnecessary portions of the strings 5 and 6 are excised, and a predetermined suture and the like are done to finish the procedure.

As described above, according to the exemplary embodiment of implant apparatus 1, since only the low invasive procedures such as punctures of the needles 16 and 17 and the sheath 15 can achieve the indwelling of the implant 2, and an incision and the like as in prior known procedures that are greatly invasive to the body may not be necessary, the burden on a patient is minimal and the patient safety is improved.

In addition, since the pad 4 and each of the strings 5 are made with separate members, the pad 4 is first inserted into the body, each of the strings 5 is inserted into the body, and thus the pad 4 can be fixed to the living body by each of the strings 5. Accordingly, the implant 2 can be easily indwelled inside the living body.

### Second Embodiment

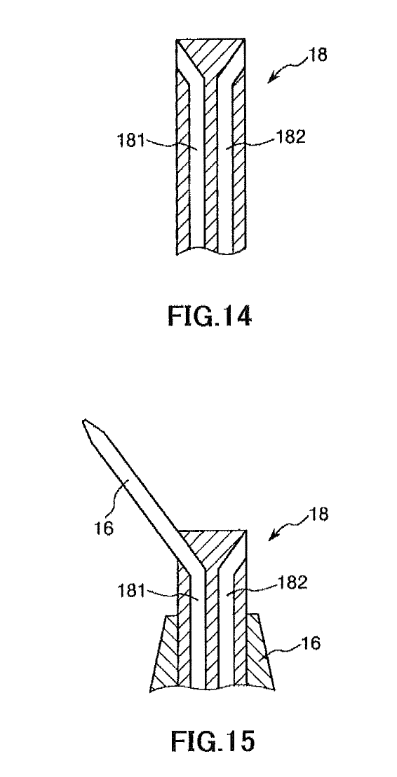

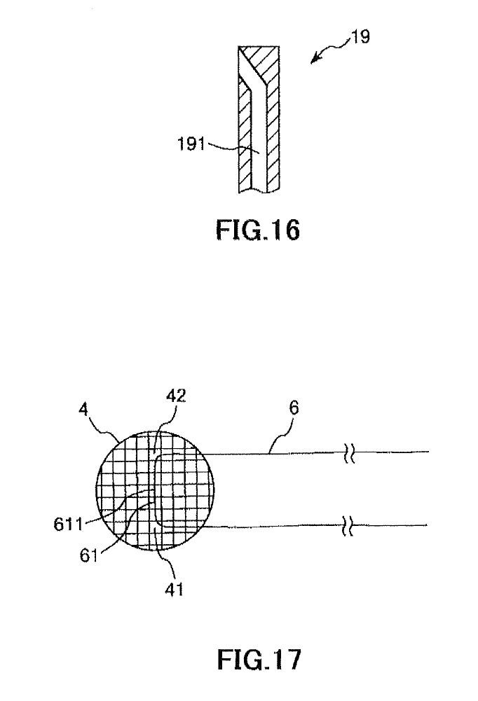

FIG. 14 is a cross-sectional view illustrating an inner tube in an implant apparatus according to a second exemplary embodiment of the disclosure. FIG. 15 is a cross-sectional view illustrating the inner tube and a needle in the implant apparatus according to the second exemplary embodiment of the disclosure. FIG. 16 is a cross-sectional view illustrating an example of another configuration of the inner tube in the implant apparatus according to the second exemplary embodiment of the disclosure.

Hereinafter, description will be given on the premise that in FIGS. 14 to 16, the upper side indicates a “distal end”, and the lower side indicates a “proximal end”.

The second exemplary embodiment will be described below with the focus on differences from the aforementioned first exemplary embodiment, and the description of the same matters will be omitted.

As illustrated in FIGS. 14 and 15, in the implant apparatus 1 according to the second exemplary embodiment, the guide apparatus 3 has an inner tube 18 that is inserted into the sheath 15 for use.

In the illustrated configuration, the shape profile of the cross-section of the inner tube 18 has a circular shape.

Two lumens 181 and 182 into which the needle 16 is inserted are formed in the inner tube 18 in such a manner that the lumens 181 and 182 are in parallel to each other along a longitudinal direction of the inner tube 18.

In addition, distal portions of the lumens 181 and 182 are bent outwardly. That is, the distal portion of the lumen 181 on the left side as shown in FIG. 14 is bent to the left side in FIG. 14, and the distal portion of the lumen 182 on the right side as shown in FIG. 14 is bent to the right side in FIG. 14.

In addition, the needle 16 has a linear shape in a free state where an external force is not applied thereto.

In the implant apparatus 1, the inner tube 18 is first inserted into the sheath 15 before the needle 16 is inserted into the sheath 15.

Subsequently, the retention portion (not shown) of the distal portion of the needle 16 retains the fixing portion 51 of one of two of the strings 5, and the needle 16 is inserted into the lumen 181 of the inner tube 18 to be moved in the direction of the distal end.

At this time, as illustrated in FIG. 15, the distal portion of the needle 16 is bent in the distal portion of the lumen 181 toward the left side of FIG. 15. Accordingly, the distal portion of the needle 16 avoids the urethra 100 and advances in the upper left direction in FIG. 15. In this way, the needle 16 can be prevented from puncturing the urethra 100.

After the needle 16 is removed, the retention portion (not shown) of the distal portion of the needle 16 retains the fixing portion 51 of the other string 5, and the needle 16 is inserted into the lumen 182 to be moved in the direction of the distal end.

At this time, the distal portion of the needle 16 is bent in the distal portion of the lumen 181 toward the right side in FIG. 15. Accordingly, the distal portion of the needle 16 avoids the urethra 100 and advances in the upper right direction in FIG. 15. In this way, the needle 16 can be prevented from puncturing the urethra 100.

According to the implant apparatus 1, the same effects as in the aforementioned first exemplary embodiment are obtained.

The configuration of the inner tube is of course not limited to the above configuration. For example, an example of another configuration of the inner tube is illustrated in FIG. 16, wherein an inner tube 19 is configured in such a manner that the inner tube 19 has a single lumen 191 and can be rotated around the axis of the sheath 15. When the inner tube 19 is used, one of two of the strings 5 is fixed and then the inner tube 19 is rotated 180° around the sheath 15.

Accordingly, the single lumen 191 has the same functions as the two lumens 181 and 182.

### Third Embodiment

FIG. 17 illustrates a pad and a string in an implant apparatus according to a third exemplary embodiment of the disclosure.

The third embodiment will be described with the focus on the differences from the aforementioned first exemplary embodiment, and the description of the same matters will be omitted.

As illustrated in FIG. 17, in the implant apparatus 1 according to the third exemplary embodiment, the string 6 is configured in such a manner that the string 6 has a foldback portion 61 which is folded back in the middle and the foldback portion 61 retains the pad 4.

In addition, in the illustrated configuration, the foldback portion 61 has a linear portion 611 which is formed in a linear shape to retain the center portion of the pad 4.

The pad 4 is thus rotationally moved around the linear portion 611 of the foldback portion 61 which is a single center of the rotary movement, and the pad 4 is not rotationally moved in other directions.

In addition, the string 6 is inserted through one mesh 41 of a pair of meshes 41 and 42 which are symmetrically arranged with respect to the center of the pad 4, the string 6 is passed through the center portion of the pad 4, the string 6 is inserted through the other mesh 42, and the foldback portion 61 is thereby caught in the line-shaped bodies defining the pad 4. Thus, the pad 4 is retained.

In a state where the string 6 is inserted into the sheath 15, both end portions of the string 6 protrude from the proximal end of the sheath 15.

When in use, the linear portion 611 of the foldback portion 61 is preferably orthogonal to the axis line of the urethral-insertion portion 7.

While both end portions of the string 6 are pulled together, the distal portion of the sheath 15 is positioned with respect to the center portion of the pad 4 similar to the first exemplary embodiment.

After the string 6 is used, the string 6 is separated and removed from the pad 4 by pulling one end portion of the string 6.

It is understood by one skilled in the art that the foldback portion 61 of the string 6 may be fixed to the pad 4.

According to the implant apparatus 1, the same effects as in the aforementioned first exemplary embodiment are obtained.

The implant and the implant apparatus according to the exemplary embodiments of the disclosure here have been described with reference to the illustrated embodiments, but the disclosure is not to be limited to the illustrated embodiments. The configuration of each portion can be replaced with any configuration having the same function.

In addition, any other components may be added to the disclosed implant and implant apparatus, and the implant and implant apparatus may be configured to arbitrarily combine two or more configurations of the exemplary embodiments within the scope of the disclosure.

When the implant is indwelled according to the disclosure here, the burden on a patient is minimal and the patient safety is improved.

In addition, since the pad and the fixing member are made with separate members, the pad can be first inserted into the body, and then the fixing member can be inserted into the body. Accordingly, the implant can be easily indwelled.

The detailed description above describes an implant and implant apparatus disclosed by way of example. The invention is not limited, however, to the precise embodiment and variations described. Various changes, modifications and equivalents can be effected by one skilled in the art without departing from the spirit and scope of the invention as defined in the accompanying claims. It is expressly intended that all such changes, modifications and equivalents which fall within the scope of the claims are embraced by the claims.

What is claimed is:

1. An implant for treatment of urinary incontinence in women comprising:
a pad, the shape of which can be recovered from a constrained state and which has a generally flat, plate-shaped appearance in a recovered state; and
a fixing member that passes through the pad to fix the pad to a living body when in use in a state where the pad supports the urethra.

2. The implant according to claim 1,
wherein the pad recovers from a folded state, a rolled-up state or a compressed state by a self-recovery force.

3. The implant according to claim 1,
wherein the pad has a mesh-like configuration, and
wherein the fixing member passes through openings in the mesh-like configuration of the pad when the fixing member fixes the pad.

4. The implant according to any one of claim 1, wherein the fixing member is a long object that is flexible.

5. The implant according to claim 4,
wherein a fixing portion that is fixed to the living body is provided in at least one end portion of the long object.

6. The implant according to claim 5,
wherein the fixing portion is a portion that punctures the living body, and the fixing portion of the long object has resistance greater than other portions of the long object when pulled out from the living body.

7. The implant according to claim 4,
wherein the pad is configured to be pulled up and fixed to a side of the urethra from a side of a vaginal wall by tension from the long object.

8. An implant apparatus comprising:
the implant according to claim 1; and
a tubular body that is used to move the pad to a target portion in a state where the shape of the pad is constrained.

9. The implant apparatus according to claim 8,
wherein the tubular body is used to move the fixing member to a target portion.

10. The implant apparatus according to claim 8, further comprising:
a positioning member that positions a distal portion of the tubular body with respect to the pad.

11. The implant apparatus according to claim 10,
wherein the positioning member is a string-like body one end side of which is fixed to the pad and is inserted into the tubular body for use.

12. The implant apparatus according to claim 11,
wherein one end side of the string-like body is fixed to a center portion of the pad, and
wherein the string-like body is configured to position the distal portion of the tubular body with respect to the center portion of the pad.

13. The implant apparatus according to claim 10,
wherein the positioning member is a string-like body that has a foldback portion which is folded back, retains the pad in the foldback portion, and is inserted into the tubular body for use.

14. The implant apparatus according to claim 13,
wherein the foldback portion of the string-like body retains the center portion of the pad, and
wherein the string-like body is configured to position the distal portion of the tubular body with respect to the center portion of the pad.

15. An implant for treatment of urinary incontinence in women and configured to be implanted in a living body, the implant comprising:
a pad having a constrained state and a recovered state, the pad having a generally flat, plate-shaped appearance in the recovered state; and
at least one fixing member configured to pass through the pad to fix the pad in position such that the pad supports the urethra when implanted.

16. The implant according to claim 15, wherein the pad is elastically deformable such that the pad returns to the recovered state from the constrained state by a self-recovery force.

17. The implant according to claim 16, wherein the pad has a mesh-like configuration defined by a plurality of openings.

18. The implant according to claim 15, wherein the at least one fixing member includes a fixing portion at one end portion thereof.

19. The implant according to claim 18, wherein the fixing portion includes a plurality of V-shaped unit fixing portions.

20. The implant according to claim 19, wherein the at least one fixing member comprises two fixing members.
